(19) United States
(12) Patent Application Publication
(10) Pub. No.: US 2011/0087013 A1
(43) Pub. Date: Apr. 14, 2011

(54) Friedel-Crafts acylation for the synthesis of aryl- and heteroaryl-(3-ethyl-4-nitrophenyl)-methanones

(76) Inventor: Bernhard Knipp, Kuerten (DE)

(21) Appl. No.: 12/897,949

(22) Filed: Oct. 5, 2010

(30) Foreign Application Priority Data
Oct. 13, 2009 (EP) 09012899.2

(51) Int. Cl. C07H 21/00; C07C 205/57; C07D 319/06; C07F 9/141

(52) U.S. Cl. 536/23.1; 568/306; 549/373; 568/13; 549/221

(57) ABSTRACT

The present invention concerns a synthesis process comprising the following steps (i) reacting 3-ethyl-4-nitrobenzoic acid with thionyl chloride to produce a 3-ethyl-4-nitrobenzoic acid chloride or a 3-ethyl-4-nitrobenzoic acid anhydride from 3-ethyl-4-nitrobenzoic acid by means of water cleavage and (ii) Friedel-Crafts acylation by reacting the 3-ethyl-4-nitrobenzoic acid chloride or the 3-ethyl-4-nitrobenzoic acid anhydride with an optionally substituted aryl-H to form an optionally substituted (3-ethyl-4-nitrophenyl)-aryl-methanone. In addition the present invention concerns compounds containing (3-ethyl-4-nitrophenyl)-aryl-methanone, characterized in that the optionally substituted aryl is an optionally substituted condensed aromate.

### RELATED APPLICATIONS

This application claims priority to European application EP 09012899.2 filed Oct. 13, 2009.

### BACKGROUND OF THE INVENTION

There have been a variety of reports about the light-directed synthesis of high density oligonucleotide microarrays using photolabile 2-(2-nitrophenyl)-propoxycarbonyl protecting groups (NPPOC) as 5′-O-carbonate esters of phosphoramidite building blocks (Stengele, K. P., and Buehler, J., Nucleosides, Nucleotides & Nucleic Acids 24 (2005) 891-896; Buehler, S., Helv. Chim. Acta 87 (2004) 620-659; WO 2004/074300). The synthesis of this benzophenone protecting group on a laboratory scale begins with the coupling of benzylcyanides to ortho-nitroethyl benzene to form the cyano oxime and subsequent exothermic Oxidative decarboxylation by treatment with hydrogen peroxide (35%) in potassium hydroxide with oxygen evolution in boiling methanol (Stengele, K. P., and Buehler, J., Nucleosides, Nucleotides & Nucleic Acids 24 (2005) 891-896; WO 2004/074300; Artini, D., et al., Arzneim. Forsch. 21 (1971) 30-36). Alternative syntheses for benzophenones of the aryl- or heteroaryl-(3-ethyl-4-nitrophenyl)-methanone type are unknown to date. With the exception of 3-ethyl-4-nitrobenzophenone (see below), no other aryl or hetero analogues of 3-ethyl-4-nitrobenzophenone have been described as a substance.

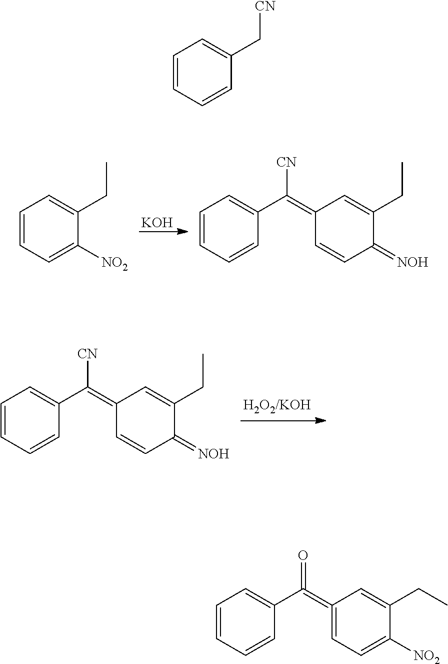

The previous synthesis occurs in two steps with moderate yields (about 26%) (WO 2004/074300). The remaining reaction products are unknown (Stengele, K. P., and Buehler, J., Nucleosides, Nucleotides & Nucleic Acids 24 (2005) 891-896; WO 2004/074300; EP 1 589 024). The oxidative decarboxylation (see above) is problematic on a plant scale with regard to safety and environmental protection. In the absence of inertization there is a risk due to continuous oxygen evolution in the highly volatile and highly flammable methanol (Stengele, K. P., and Buehler, J., Nucleosides, Nucleotides & Nucleic Acids 24 (2005) 891-896; WO 2004/074300; EP 1 589 024) of fire, deflagration and under certain circumstances explosions if peroxides are formed from hydrogen peroxide (see above). In addition in the case of incomplete oxidative decarboxylation, the generation of toxic cyanides in sewage water (KCN) and discharged air (HCN, dicyanogen) has to be assessed. The general synthetic approach is per se limited.

Hence, the object of the present invention is to provide an improved process for producing phosphoramidites with a photolabile NPPOC protecting group and the production of new previously undescribed photolabile NPPOC protecting groups as phosphoramidite building blocks.

### BRIEF DESCRIPTION OF THE INVENTION

Hence, the present invention concerns a synthesis process comprising the following steps
a) reacting 3-ethyl-4-nitrobenzoic acid with a thionyl halide (preferably thionyl chloride) to produce a 3-ethyl-4-nitrobenzoic acid halide
b) Friedel-Crafts acylation by reacting the 3-ethyl-4-nitrobenzoic acid halide with an optionally substituted aryl-H to form an optionally substituted (3-ethyl-4-nitrophenyl)-aryl-methanone.

Aryl-His preferably benzene or an aromate which is optionally substituted or preferably a condensed aromate compound which is optionally substituted. A typical example is naphthalene.

According to the invention the optionally substituted (3′-ethyl-4-nitrophenyl)aryl-methanones can be ketalized in a further step c) with glycol or 1,3-propanediol (Stengele, K. P., and Buehler, J., Nucleosides, Nucleotides & Nucleic Acids 24 (2005) 891-896) to, protect the carbonyl group. The dioxolanes or dioxanes that result from this process lead to [3-(2-hydroxy-1-methyl-ethyl)-4-nitrophenyl]-aryl-methanones by reaction with Triton-B/paraformaldehyde (Stengele, K. P., and Buehler, J., Nucleosides, Nucleotides & Nucleic Acids 24 (2005) 891-896) and subsequent deprotection with HCl/water (Stengele, K. P., and Buehler, J., Nucleosides, Nucleotides & Nucleic Acids 24 (2005) 891-896).

The [3-(2-hydroxy-1-methyl-ethyl)-4-nitrophenyl]-aryl-methanone that is formed can be used as a starting material for the synthesis of a nucleoside containing a photolabile NPPOC protecting group (Stengele, K. P., and Buehler, J., Nucleosides, Nucleotides & Nucleic Acids 24 (2005) 891-896; Buehler, S., Helv. Chim. Acta 87 (2004) 620-659; WO 2004/074300). The corresponding nucleoside can then be converted into a phosphoramidite with a photolabile NPPOC protecting group (WO 2004/074300).

The present invention also concerns the substances (3-ethyl-4-nitrophenyl)-aryl-methanone or [3-(2-hydroxy-1-methyl-ethyl)-4-nitrophenyl)]-aryl-methanone characterized in that the optionally substituted aryl preferably is a condensed aromate compound, which is optionally substituted. The present invention also concerns compounds which contain corresponding substituents. In the context of the present invention, the term “condensed aromate compound relates to any aromatic compound, which comprises at least two homocyclic or heterocycleic aromatic ring structures.

In particular the compounds according to the present invention are nucleosides which contain a [3-(2-O-1-methyl-ethyl)-4-nitrophenyl]-aryl-methanone substituted at the 5′ or 3′ position. This substituent is preferably coupled to the nucleoside via an O-carbonate ester. Corresponding nucleoside phosphoramidites are also preferred in which the phosphoramidite group is located at the 3′ or 5′ position at which no [3-(2-O-1-methyl-ethyl)-4-nitrophenyl]-aryl-methanone is substituted.

The condensed aromate of all said compounds is preferably naphthalene.

### FIGURES

FIG. 1. Schematic representation of synthetic pathways according to the invention in a general form (middle line) as well as for the special reaction with benzene (upper line) or naphthalene (lower line).

### DETAILED DESCRIPTION OF THE INVENTION

The present invention concerns a new process for preparing precursors of photolabile NPPOC protecting groups such as for example (3-ethyl-4-nitrophenyl)-phenyl-methanone which is also referred to in professional circles as 3-ethyl-4-nitrobenzophenone. The process according to the invention additionally allows the preparation of precursors which it has not been previously possible to synthesize from which previously unknown NPPOC derivatives can be prepared.

The process according to the invention for the preparation of protecting groups of the NPPOC type is essentially based on two successive steps. In the first step a halogenation and preferably a chlorination of commercially available 3-ethyl-4-nitrobenzoic acid takes place to form the intermediate compound 3-ethyl-4-nitrobenzoic acid chloride which is previously unknown in the prior art.

Then in a second step a Friedel-Crafts acylation is then used to substitute the chlorine atom by any optionally substituted aryl which still has at least one H atom so that this is referred to in the following as aryl-H. (3-Ethyl-4-nitrophenyl)-aryl-methanone is formed in this process.

As shown by the examples the production process according to the invention can be carried out without any problems in a one-pot process with 85% yield and it can be carried out industrially on a large scale.

If the aryl is benzene, then 3-ethyl-4-nitro-benzophenone is formed or according to IUPAC nomenclature (3-ethyl-4-nitrophenyl)-phenyl-methanone.

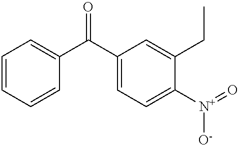

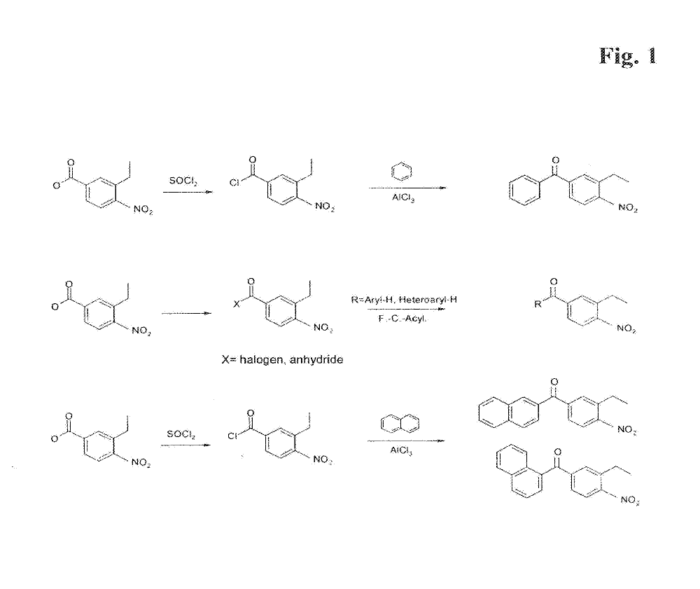

The corresponding synthesis process is shown schematically in the upper line of FIG. 1. Commercially available 3-ethyl-4-nitrobenzoic acid is converted in the first step to 3-ethyl-4-nitrobenzoic acid chloride by'reaction with thionyl chloride. This is followed by a Friedel-Crafts acylation with benzene in the presence of aluminium chloride.

The middle line of FIG. 1 shows a general form of the synthesis process according to the invention. In each case the synthesis begins with 3-ethyl-4-nitrobenzoic acid which in the first step is converted by suitable methods to form the corresponding acid halide and can subsequently be acylated with any aryl with the aid of a Friedel-Crafts acylation. The aryl which is used in each case can be substituted in any manner except for one position.

Instead of reacting 3-ethyl-4-nitrobenzoic acid with a thionyl halide, a 3-ethyl-4-nitrobenzoic acid anhydride can be produced as the first step in the synthesis from 3-ethyl-4-nitrobenzoic acid by removal of water. Such anhydrides can also be subjected to a Friedel-Crafts acylation in an analogous manner.

Since the process according to the invention shows a general synthetic route to the aryl or hetero analogue of 3-ethyl-4-nitrobenzophenone which are substances which it has previously not been possible to synthesize, one aspect of the invention also refers to special substances containing a (3-ethyl)-4-nitrophenyl)-aryl-methanone structure which is characterized in that the optionally substituted aryl residue is an optionally substituted condensed aromate.

In this connection the condensed aromate can consist of 2-5 of any homocyclic or heterocyclic ring systems where each ring independently of one another either forms a hexycycle or a pentacycle.

A particularly preferred embodiment prepares (3-ethyl-4-nitrophenyl)-naphthalene-1-yl-methanone:

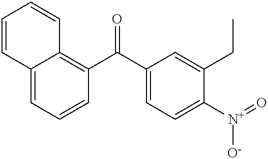

The synthesis is shown schematically in the lower line of FIG. 1. In this process in principle two different positional isomers i.e. (3-ethyl-4-nitrophenyl)-naphthalene-1-yl-methanone or (3-ethyl-4-nitrophenyl)-naphthalene-2-yl-methanone can be formed in the Friedel-Crafts acylation of 3-ethyl-4-nitrobenzoic acid chloride with naphthalene where it is possible to steer towards the preferred formation of (3-ethyl-4-nitrophenyl)-naphthalene-1-yl-methanone by carrying out the acylation at the lowest possible temperatures. In this connection the 1-isomer is preferably isolated by appropriate crystallization methods known to a person skilled in the art. In contrast (3-ethyl-4-nitrophenyl)-naphthalene-2-yl-methanone is preferably formed at higher temperatures. In this case the 2-isomer is preferably isolated by the column chromatographic methods known to a person skilled in the art.

Moreover, the (3-ethyl-4-nitrophenyl)-aryl-methanones formed according to the invention can be converted with the aid of suitable synthesis processes into [3-(2-hydroxy-1-methyl-ethyl)-4-nitrophenyl]-aryl-methanones (Stengele, K. P., and Buehler, J., Nucleosides, Nucleotides & Nucleic Acids 24 (2005) 891-896; Buehler, S., Helv. Chim. Acta 87 (2004) 620-659; WO 2004/074300). In a first step they are reacted with suitable diols to produce dioxolanes or dioxanes which are firstly admixed with Triton B/paraformaldehyde and subsequently with HCl/water (Stengele, K. P., and Buehler, J., Nucleosides, Nucleotides & Nucleic Acids 24 (2005) 891-896; Buehler, S., Helv. Chim. Acta 87 (2004) 620-659; WO 2004/074300).

This is elucidated in more detail in the following using (3-ethyl-4-nitrophenyl)-naphthalene-1-yl-methanone as an example.

In a first embodiment (3-ethyl-4-nitrophenyl)-naphthalene-1-yl-methanone is for example firstly admixed with glycol. 2-(3-Ethyl-4-nitrophenyl)-2-naphthalene-1-yl-[1,3]dioxolane is formed in this process.

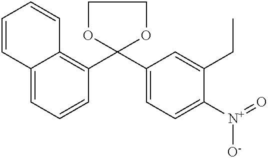

Subsequently the 2-(3-ethyl-4-nitrophenyl)-2-naphthalene-1-yl-[1,3]dioxolane is hydroxymethylated in the presence of Triton B/paraformaldehyde to form 2-[5-(2-naphthalene-1-yl-[1,3]dioxolan-2-yl)-2-nitrophenyl]-propan-1-ol.

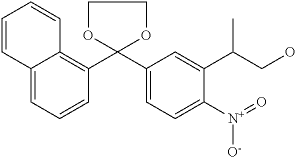

After treatment with HCl/water [3-(2-hydroxy-1-methyl-ethyl)-4-nitrophenyl]-naphthalene-1-yl-methanone is finally formed.

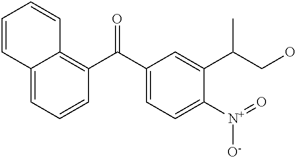

In an alternative embodiment (3-ethyl-4-nitrophenyl)-naphthalene-1-yl-methanone is for example firstly admixed with 1,3-propanediol. 2-(3-Ethyl-4-nitrophenyl)-2-naphthalene-1-yl-[1,3]dioxane is formed in this process.

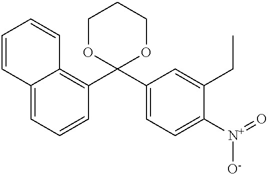

Subsequently the 2-(3-ethyl-4-nitrophenyl)-2-naphthalene-1-yl-[1,3]dioxane is hydroxymethylated also in the presence of Triton B/paraformaldehyde to form (2-[5-(2-naphthalene-1-yl-[1,3]dioxane-2-yl)-2-nitrophenyl]-propan-1-ol.

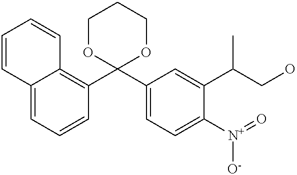

After treatment with HCl/water [3-(2-hydroxy-1-methyl-ethyl)-4-nitrophenyl]-naphthalene-1-yl-methanone is then also formed.

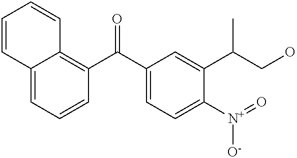

The substances according to the invention formed with the aid of the synthesis described above, are photolabile protecting groups or precursors of the so-called NPPOC class which are characterized by a 2-(2-nitrophenyl)ethyl skeletal structure. This structure can be coupled to nucleosides and subsequently converted into nucleoside phosphoramidites. Hence, such phosphoramidites contain protecting groups which can be cleaved by photolysis.

The present invention therefore also concerns nucleosides and nucleoside phosphoramidites which contain (3-ethyl-4-nitrophenyl)-aryl-methanone or [3-(2-hydroxy-1-methyl-ethyl)-4-nitrophenyl]-aryl-methanone protecting groups which are characterized in that the optionally substituted aryl is an optionally substituted condensed aromate. In this connection they are particularly preferably phosphoramidites containing (3-ethyl-4-nitrophenyl)-naphthalene-1-yl-methanone or [3-(2-O-1-methyl-ethyl)-4-nitro-phenyl]-naphthalene-1-yl-methanone.

The preparation of nucleosides and nucleoside phosphoramidites containing the photolabile protecting groups according to the invention is carried out by standard coupling methods known to a person skilled in the art as described for example in (Stengele, K. P., and Buehler, J., Nucleosides, Nucleotides & Nucleic Acids 24 (2005) 891-896; Buehler S., Helv. Chim. Acta 87 (2004) 620-659; WO 2004/074300; WO 97/44345). In this process the alcohol [3-(2-hydroxy-1-methyl-ethyl)-4-nitrophenyl]-aryl-methanone is firstly converted into the corresponding chloroformic acid ester with the aid of phosgene or derivatives thereof such as diphosgene or triphosgene (WO 2004/074300).

Subsequently the coupling to the respective nucleoside or nucleoside derivative is carried out. Phosphoramidite nucleoside derivatives are preferably prepared therefrom because the derivatives that are subsequently formed and provided with a protecting group can be used directly as building blocks for conventional oligonucleotide synthesis.

Phosphoramidite nucleoside derivatives can be, prepared by methods known to a person skilled in the art by reacting nucleosides with phosphanes (earlier nomenclature: phosphines) in the presence of tetrazole (Stengele, K. P., and Buehler, J., Nucleosides, Nucleotides & Nucleic Acids 24 (2005) 891-896; WO 2004/074300). As a rule 3′ phosphoramidites are prepared because they can be used for conventional oligonucleotide synthesis in the 3′-5′ orientation. Alternatively 5′ phosphoramidites are produced which can be used for an inverse oligonucleotide synthesis.

The chloroformic acid esters that are prepared can react with the free hydroxyl group of nucleosides or nucleoside derivatives to form carbonic acid 2-[5-(arylene carbonyl)-2-nitrophenyl]-propyl esters or phosphoramidites thereof. The latter can then be used directly in the oligonucleotide synthesis as photoactivatable building blocks.

### EXAMPLES

### Example 1

### (3-Ethyl-4-nitrophenyl)-phenyl-methanone (═3-ethyl-4-nitrobenzophenone)

7 g (36 mmol) 3-ethyl-4-nitrobenzoic acid was boiled under reflux in 15 ml (205 mmol) thionyl chloride for 30 min while stirring (until no more gas was generated). Afterwards excess thionyl chloride was removed by distillation under a vacuum at 50° C., the residue of evaporation was dissolved in 20 ml (225 mmol) benzene and 6.5 g (49 mmol) aluminium chloride was added in portions. The mixture was boiled under reflux for 2.5 h while stirring, subsequently cooled to room temperature and poured into 75 g ice water. The aqueous phase was extracted twice with 25 ml ethyl acetate in each case, the organic phases were concentrated and the residue was recrystallized from ethanol containing activated charcoal. Yield: 7.9 g (85% of theory); light yellow crystals; FP: 64-65° C.; purity: 99% (HPLC); NMR and mass spectroscopy: correspond.

### Example 2

### (3-Ethyl-4-nitrophenyl)-phenyl-methanone (═3-ethyl-4-nitrobenzophenone)

35 g (0.18 mol) 3-ethyl-4-nitrobenzoic acid was boiled under reflux in 75 ml (1.03 mol) thionyl chloride for 30 min while stirring (until no more gas was generated). Afterwards excess thionyl chloride was removed by distillation under a vacuum at 50° C., the residue of evaporation was dissolved in 50 ml (0.57 mol) benzene and added dropwise within 10 minutes to a mixture of 32.5 g (0.25 mol) aluminium chloride in 50 ml (0.57 mol) benzene. The mixture was boiled for 2.5 h under reflux while stirring, subsequently cooled to room temperature and poured into 375 g ice water. 10 ml concentrated. HCl was added to the aqueous phase which was subsequently extracted twice with 150 ml ethyl acetate each time; the organic phases were washed twice with 75 ml water each time, concentrated and the residue was recrystallized from ethanol containing active charcoal.

Yield: 36.7 g (80% of theory); light yellow crystals; FP: 63-64° C.

### Example 3

### 3-Ethyl-4-nitrophenyl)-naphthalene-1-yl-methanone

7.0 g (36 mmol) 3-ethyl-4-nitrobenzoic acid was boiled under reflux in 15 ml (205 mmol) thionyl chloride for 30 min while stirring (until no more gas was generated) and afterwards excess thionyl chloride was removed by distillation under a vacuum at 50° C. The residue of evaporation and 4.5 g (35 mmol) naphthalene were dissolved in 35 ml dichloromethane, cooled to −40° C. and 6.5 g (49 mmol) aluminium chloride was added in portions within one hour. The mixture was stirred for 30 min at −40° C., 2 h at −20° C., poured into 300 g ice water and extracted with 80 ml dichloromethane. The organic phase was washed with 100 ml water and with 100 ml saturated sodium hydrogen carbonate solution, concentrated and the residue was recrystallized from ethanol. Yield: 7.7 g (70% of theory); light yellow crystals; FP: 57-58° C.; purity: 99% (HPLC); NMR and mass spectroscopy: corresponds.

### Example 4

### 2-(3-Ethyl-4-nitrophenyl)-2-naphthalene-1-yl-[1,3]-dioxolane

7.6 g (25 mmol) (3-ethyl-4-nitrophenyl)-naphthalene-1-yl-methanone, 1.2 g (6 mmol) p-toluenesulphonic acid and 29 ml (520 mmol) glycol in 41 ml toluene were boiled for 24 hours while stirring on a water separator. The mixture was subsequently washed with 17 ml 2% sodium hydroxide solution and twice with 22 ml saturated saline solution in each case, concentrated and the residue was recrystallized from methanol. Yield: 7.6 g′ (87% of theory); light yellow crystals; FP: 88-90° C.; NMR and mass spectroscopy: corresponds.

### Example 5

### 2-[5-(2-naphthalene-1-yl-11,31-dioxolan-2-yl)-2-nitrophenyl]-propan-1-ol

7.0 g (20 mmol) 2-(3-ethyl-4-nitrophenyl)-2-naphthalene-1-yl-[1,3]dioxolane, 2.4 g (27 mmol) paraformaldehyde and 30 ml DMSO were stirred with 6.4 Triton B (35% in methanol) for 3 hours at 90° C. Subsequently 35 ml dichloromethane and 60 ml, water were added, the mixture was extracted and the organic phase was washed twice with 40 ml water in each case, concentrated and the residue was re-crystallized from diisopropyl ether. Yield: 7.0 g (92% of theory); light-yellow crystals; FP: 115° C.; NMR and mass spectroscopy: corresponds.

### Example 6

### [3-(2-Hydroxy-1-methyl-ethyl)-4-nitrophenyl]-naphthalene-1-yl-methanone

4.3 ml Concentrated HCl was added to 6.8 g (18 mmol) 2-[5-(2-naphthalene-1-yl-[1,3]dioxolan-2-yl-nitrophenyl]-propan-1-ol dissolved in 22 ml ethanol and boiled under reflux for 2.5 hours while stirring. Subsequently 25 ml dichloromethane and 50 ml water were added, the mixture was extracted and the organic phase was washed twice with 38 ml water in each case, dried and the solvent was removed under a vacuum. Yield: 6 g (quantitative); viscous yellow oil; NMR and mass spectroscopy: corresponds.

1. A synthesis process comprising the following steps
a) reacting 3-ethyl-4-nitrobenzoic acid with a thionyl halide to produce a 3-ethyl-4-nitrobenzoic acid halide; and
b) Friedel-Crafts acylation by reacting the 3-ethyl-4-nitrobenzoic acid chloride or the 3-ethyl-4-nitrobenzoic acid anhydride with an optionally substituted aryl-H to form an optionally substituted (3-ethyl-4-nitro-phenyl)-aryl-methanone.

2. The process according to claim 1, wherein the 3-ethyl-4-nitrobenzoic acid is reacted with thionyl chloride.

3. The process according to claim 1 wherein the aryl-His benzene.

4. The process according to claim 2, wherein the aryl-H is benzene.

5. The process according to claim 1, wherein aryl-His an optionally substituted condensed aromate.

6. The process according to claim 5, wherein the aryl-H is naphthalene.

7. The process according to claim 1, further comprising step c) reacting the product formed with glycol or 1,3-propanediol.

8. The process according to claim 7, wherein the product formed in step c) is converted into [3-(2-hydroxy-1-methyl-ethyl)-4-nitrophenyl]-aryl methanone in a subsequent step d) firstly with Triton B/paraformaldehyde and subsequently with HCl/water.

9. The process according to claim 8, further comprising the step of synthesizing a nucleoside containing a photolabile protecting group using the [3-(2-hydroxy-1-methyl-ethyl)-4-nitrophenyl]-aryl methanone.

10. The process according to claim 9, further comprising the step of converting the nucleoside containing a photolabile protecting group into a phosphoramidite.

11. A compound of the formula (3-Ethyl-4-nitrophenyl)-aryl-methanone or [3-(2-hydroxy-1-methyl-ethyl)-4-nitrophenyl]-aryl-methanone, wherein the optionally substituted aryl is a condensed aromate, which is optionally substituted.

12. A nucleoside containing a substituent according to claim 10 at the 5′ or 3′ position, said substituent being a [3-(2-O-1-methyl-ethyl)-4-nitrophenyl]-aryl-methanone.

13. The nucleoside according to claim 12, wherein the substituent is coupled to the nucleoside via a carbonate ester group.

14. The nucleoside according to claim 13, which has a phosphoramidite group which is located at that free 3′ or 5′ position which contains no [3-(2-O-1-methyl-ethyl)-4-nitrophenyl]-aryl methanone substituent.

15. A compound according to claim 11, wherein the condensed aromate is naphthalene.
